(19) United States
(12) Plant Patent Application Publication
(10) Pub. No.: US 2019/0014705 P1
(43) Pub. Date: Jan. 10, 2019

(54) Lupinus 'LSDRW03-D'

(50) ;

(71) Applicant: Kazunori Sato, Tokyo (JP)

(72) Inventor: Kazunori Sato, Tokyo (JP)

(21) Appl. No.: 15/998,134

(22) Filed: Jul. 5, 2018

Related U.S. Application Data
(60) Provisional application No. 62/604,472, filed on Jul. 8, 2017.

(51) Int. Cl. A01H 6/54

(52) U.S. Cl. PLT/263.1

(57) ABSTRACT

A new and distinct Lupinus cultivar named ‘LSDRW03-0’ is disclosed, characterized by distinctive bright yellow, fully double, strongly fragrant flowers. Plants uniquely bloom under both short day and long day conditions without vernalization. The new cultivar is a Lupinus, suitable for ornamental purposes.

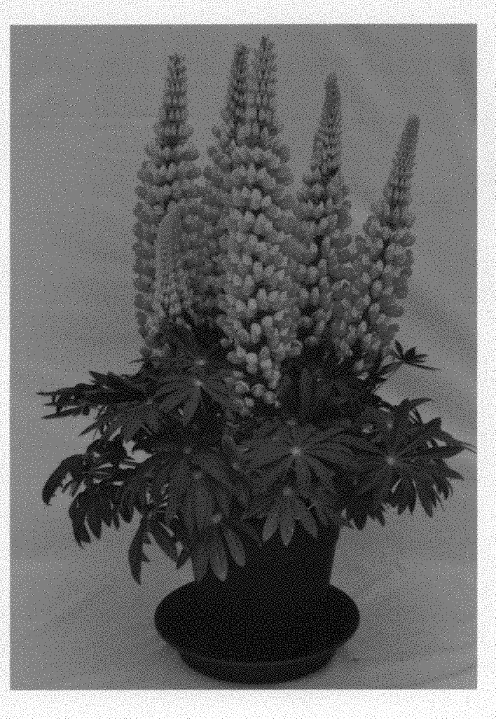

Latin name of the genus and species: Lupinus hybrida.

Variety denomination: ‘LSDRW03-0’.

### BACKGROUND OF THE INVENTION

new variety, hereinafter referred to as ‘LSDRW03-0’, was discovered as part of a planned breeding program. The intent of the breeding program was to develop Lupine hybrids for commercial ornamental applications.

### SUMMARY OF THE INVENTION

The cultivar ‘LSDRW03-0’ has not been observed under all possible environmental conditions. The phenotype may vary somewhat with variations in environment such as temperature, day length, and light intensity, without, however, any variance in genotype. The following traits have been repeatedly observed and are determined to be the unique characteristics of ‘LSDRW03-0’ These characteristics in combination distinguish ‘LSDRW03-0’ as a new and distinct Lupinus cultivar:
1. Plants uniquely bloom under both short day and long day conditions without vernalization.
2. Pink and white flower coloration.

### COMPARISON TO PARENT VARIETY

‘LSDRW03-0’ is similar in most horticultural characteristics to the seed parent variety. Plants of the new cultivar ‘LSDRW03-0’ however, differ in the following characteristics:
1. The new variety has a different flower color.

‘LSDRW03-0’ is similar in most horticultural characteristics to the pollen parent variety. Plants of the new cultivar ‘LSDRW03-0’ however, differ in the following characteristics:
1. The new variety has a different flower color.

### COMMERCIAL COMPARISON

‘LSDRW03-0’ can be compared to the commercial variety Lupinus ‘West Country Inspiration’. unpatented. Plants of the new cultivar ‘LSDRW03-0’ are similar to plants of ‘West Country Inspiration’ in most horticultural characteristics, however, plants of the new cultivar ‘LSDRW03-0’ differ in the following:
1. Plants of the new variety produces more inflorescence per plant, as well as wider inflorescence.
2. Plants of the new variety are shorter and sturdier.
3. Plants of the new variety will begin flowering without vernalization, as required by this comparator.

‘LSDRW03-0’ can be compared to the commercial variety Lupinus ‘West Country Blossom’, unpatented. Plants of the new cultivar ‘LSDRW03-0’ are similar to plants of ‘West Country Blossom’ in most horticultural characteristics, however, plants of the new cultivar ‘LSDRW03-0’ differ in the following:
1. Flower of the new variety medium pink with white, flowers of this comparator are very light pink with white.
2. Plants of the new variety are shorter and sturdier.
3. Plants of the new variety will begin flowering without vernalization, as required by this comparator.

### BRIEF DESCRIPTION OF THE PHOTOGRAPH

The accompanying photograph in illustrates in full color a typical plant of ‘LSDRW03-0’ grown in a greenhouse in Santa Paula, Calif. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007 except where general terms of ordinary dictionary significance are used. The following observations and measurements describe ‘LSDRW03-0’ plants grown outdoors in Santa Paula, Calif. Plants are approximately 1 year old, in a 2-gallon nursery container. Measurements and numerical values represent averages of typical plant types.
Botanical classification: Lupinus hybrida ‘LSDRW03-0’.
Propagation: Typically by shoot tissue culture.
Root description: Single main root, moderate branching of short lateral roots. Cream to tan in color, not accurately measured with an R.H.S. chart.
Plant:
   Growth habit.—Compact and herbaceous vigorous perennial. Mounding foliar base topped with upright raceme inflorescences.
   Height.—Approximately 20 cm to top of foliar plane, approximately 48 cm to top of flowering plane.
   Plant spread.—Approximately 35 cm.
   Branching characteristics.—No true branching. Leaves emerge direct from base of plant, or occur directly on the flowering stem.
   Number of basal clumps of leaves.—Average 6 to 8 per plant.
   Number of leaves per clump.—Average 12 to 16.
   Age of plant described.—One year.
Foliage:
   Leaf.—Palmately compound. Occuring as basal whorls, or opposite or alternate along flowering stem. Shape: Orbicular. Length: 12.0 cm. Diameter: 11.5 cm. Quantity: Approximately 20 to 30 basal leaves, per plant. Average range 8 to 14 floral stem leaves.
   Leaflets.—Quantity: Average range 9 to 12. Shape: Oblanceolate. Arrangement: Radial. Average Length: Longest leaflet per leaf average 6.5 cm. Shortest leaflet per leaf average 4.7 cm. Average Width: Central leaflet average 1.6 cm. Apex: Acute to nearly acuminate. Base: Attenuate. Margin: Entire. Texture of top surface: Glabrous. Texture of bottom surface: Glabrous. Appearance of top surface: Matte. Appearance of bottom surface: Matte. Aspect: Flat to very slightly reflexed downward. Color: Upper side: Near RHS Green 137B. Under side: Near RHS Green 138A. Venation: Type: Pinnate. Venation color upper side: Near RHS Yellow-Green 145A. Venation color under side: Near RHS Yellow-Green 145C. Petiole: Length. Average range 8.0 to 11.0 cm. Diameter: 0.4 cm. Color: Near RHS Yellow-Green 144D. Texture: Minutely hirsute.
Flower:
   Natural flowering season.—Spring through Summer.
   Inflorescence type and habit.—Single large papilonaceous flowers evenly and symmetrically arranged on racemes.
   Flower longevity on plant.—Individual flowers approximately 15 days on the plant, not persistent.
   Quantity of flowers.—40 to 120 flowers and buds per inflorescence. Typically 4 to 5 inflorescences per plant at one time.
   Inflorescence size.—Diameter: Approximately 7.5 cm. Height: Average range 20 to 45 cm.
   Flower size (excluding pedicel).—Length: Approximately 1.9 cm. Height: Average range 2.0 cm.
   Peduncle.—Length: Approximately 7.0 cm from uppermost leaf to lowermost flower. Diameter: Approximately 0.7 cm. Color: Near RHS Yellow-Green 145A. Strength: Flexible, moderately strong. Texture: Short pubescence.
   Pedicel.—Length: Approximately 1.0 to 1.5 cm. Diameter: Approximately 0.15 cm. Color: Near RHS Green 138D, flushed Purple N77C. Strength: Strong. Texture: Softly pubescent.
   Petals.—Quantity and arrangement: 1 upper banner, 2 lateral “wings” and a lower keel. Banner: Length: Approximately 2.0 cm. Width (furled, natural state): Approximately 1.0 cm. Shape: Orbicular, when unfurled. Margin: Entire. Apex: Mucronate. Base: Obtuse. Texture: Smooth, glabrous all surfaces. Aspect: Folded, margins furled. Banner Color: When opening: Outer surface: Upper section near RHS Yellow-Green 150D, mid-section Red-Purple 65D, basal blotch near Red-Purple 71A. Inner surface: Near RHS Red-Purple 65D, basal blotch near Red-Purple 71A. Fully opened: Outer surface: Near RHS White N155D, base blotched Red-Purple 70A. Inner surface: Near RHS White N155D, base blotched Red-Purple 70A. Fading: Outer surface: Near RHS White N155D, base blotched Red-Purple 70A and 70C. Inner surface: Near RHS White N155D, base blotched Red-Purple 70A and 70C. Wing: Length: Approximately 1.7 cm. Width): Approximately 1.0 cm. Shape: Ovate. Margin: Entire. Apex: Rounded, frequently fused to opposite wing at apex. Base: Truncate. Texture: Smooth, glabrous all surfaces. Aspect: Inwardly cupped. Wing Color: When opening: Outer surface: Near RHS Yellow-Green 150D. Inner surface: Near RHS Red-Purple 65D. Fully opened: Outer surface: Near RHS Red-Purple 71D. Inner surface: Near RHS Red-Purple 73A. Fading: Outer surface: Near RHS Red-Purple 73A, streaked N74B. Inner surface: Near RHS Red-Purple N74B. Keel: Length: Approximately 1.4 cm. Width): Approximately 0.7 cm. Shape: Scythe. Margin: Entire. Apex: Awn-like. Base: Truncate. Texture: Smooth, glabrous all surfaces. Aspect: Folded. Wing Color: When opening: Outer surface: Near RHS Yellow-Green 150D, apex Purple N79B. Inner surface: Near RHS Yellow-Green 150D, apex Purple N79C. Fully opened: Outer surface: Near RHS White 155D, apex Purple 77A, very tip of apex Purple N77A. Inner surface: Near RHS Purple 75D, apex Purple 77A, very tip of apex Purple N77A. Fading: Outer surface: Near RHS Purple 75D, apex Purple 77A, very tip of apex Purple N77A. Inner surface: Near RHS Purple 75D, apex Purple 77A, very tip of apex Purple N77A.
   Bud.—Shape: Flattened ovate. Length: Approximately 1.5 cm. Diameter: Approximately 0.8 cm. Color: Near RHS Red-Purple 65D. Sepals: Quantity: 2, one fully developed, one minute (less than 1 mm in length), sometimes missing. Length: Approximately 5 mm. Width: Approximately 4 mm. Shape: Deltate. Aspect: Cupped. Apex: Mucronate. Margin: Entire. Color. Interior Surface: RHS Yellow-Green 144D. Exterior Surface: RHS Yellow-Green 144C. Texture: Glabrous. Fragrance: Strong, sweet scent.
Reproductive organs:
   Stamens.—Number: Approximately 8. Filament length: Approximately 0.5 cm. Filament color: Near RHS Yellow-Green 150D.
   Anthers.—Length: 0.3 cm. Shape: Elongated ovoid. Color: Near RHS Yellow-Orange 23A. Pollen: Scant, colored near Yellow-Orange 23A.
   Pistil.—Number: 1. Length: Approximately 1.5 cm. Stigma: Shape: Globular. Color: Near RHS Yellow-Orange 18B. Ovary: Near RHS Green 141D, approximately 2 mm in diameter.
Other characteristics:
   Disease and pest resistance.—Not observed to be susceptible nor resistant to normal diseases and pests of Lupinus.
   Temperature tolerance.—Typically USDA Zones 3 to 9.
   Fruit/seed production.—Not observed.

What is claimed is:

1. A new and distinct cultivar of Lupinus plant named ‘LSDRW03-0’ as herein illustrated and described.
